(19) United States
(12) Patent Application Publication
(10) Pub. No.: US 2012/0203312 A1
(43) Pub. Date: Aug. 9, 2012

(54) SURGICAL COOLING DEVICE

(76) Inventors: Rachel Elizabeth Batzer, Jacksonville, OR (US); Thomas Michael Cervantes, Cambridge, MA (US); Nadeem Nizar Dhanani, Boston, MA (US); Raymond Anthony Lewis, Valley Stream, NY (US); Christina Margaret Simpson, Boston, MA (US); Julia Anne Stark, Somerville, MA (US); Edward Kurt Summers, Cambridge, MA (US)

(21) Appl. No.: 13/347,178

(22) Filed: Jan. 10, 2012

Related U.S. Application Data
(60) Provisional application No. 61/439,849, filed on Feb. 5, 2011.

(51) Int. Cl. A61F 7/12

(52) U.S. Cl. 607/105

(57) ABSTRACT

A surgical device controls the temperature of an organ such as a kidney during minimally invasive surgery. The device includes a number of chambers fed by a fluid port. At least one of the chambers includes one or more baffles so that a cooling fluid entering through the fluid port is selectively distributed in a desired manner about an enclosed organ. The device may be fabricated of materials that can be rolled, folded, or otherwise compressed into a form suitable for delivery to a minimally-invasive surgical site.

### CROSS-REFERENCE TO RELATED APPLICATIONS

This application claims the benefit U.S. Provisional Patent Application No. 61/439,849 filed on Feb. 5, 2011, which is hereby incorporated by reference in its entirety.

### FEDERALLY SPONSORED RESEARCH

This invention was made with Government support under Grant No. W81XWH-09-2-0001 awarded by the U.S. Army Medical Research and Materiel Command. The Government has certain rights in this invention.

### FIELD OF THE INVENTION

The invention generally relates to methods and systems for cooling an organ such as a kidney during a minimally invasive surgical procedure.

### BACKGROUND

In surgical procedures such as a laparoscopic partial nephrectomy, blood flow to the kidney is occluded by clamping the renal artery and vein to prevent excessive bleeding, resulting in warm ischemia (lack of blood flow) to the kidney. Permanent damage can occur to the kidney if this condition lasts for more than thirty minutes. Thus, the ability to perform more complex and time-consuming procedures with minimally-invasive techniques remains constrained.

In an open surgical context, a mild hypothermia can mitigate the effects of warm ischemia. So, for example, cooling the kidney to about 20° C. can extend ischemic time up to 2.5 hours. In open surgery, this cooling can be obtained by packing crushed saline ice around the kidney. However, no similarly effective cooling method exists for minimally invasive surgery. At the same time, minimally invasive surgery is often a preferred option for patients due to its many benefits including reduced blood loss, shorter hospitalization and recovery time, reduced risk of infection, and reduced scar tissue.

Existing techniques for cooling in a minimally invasive context suffer from a variety of disadvantages. Some are too complicated or cumbersome to manipulate in the body. Some provide limited contact surfaces with the organ resulting in impaired cooling and increased patient risk. Some devices employ numerous channels for circulating a cold sterile fluid, but such devices can fail to maintain consistent pressurization and flow, resulting in uneven cooling.

There remains a need for an improved device and method to cool an organ in a minimally invasive procedure.

### SUMMARY

A surgical device controls the temperature of an organ such as a kidney during minimally invasive surgery. The device includes a number of chambers fed by a fluid port. At least one of the chambers includes one or more baffles so that a cooling fluid entering through the fluid port is selectively distributed in a desired manner about an enclosed organ. The device may be fabricated of materials that can be rolled, folded, or otherwise compressed into a form suitable for delivery to a minimally-invasive surgical site.

In one embodiment, a first chamber may have a first inner surface and a first outer surface thermally insulated to deter heat flow through the first outer surface. A second chamber may have a second inner surface facing the first inner surface and a second outer surface thermally insulated to deter heat flow through the second outer surface. The one or more baffles in the second chamber may mechanically couple the second inner surface to the second outer surface in order to restrict expansion of the second chamber beyond a predetermined volume, at which predetermined volume additional fluid received through the fluid port will be forcibly directed into the first chamber.

The first chamber and the second chamber may be shaped and sized to envelop a human organ such as a kidney between the first inner surface and the second inner surface thereof during a surgical procedure.

The device may further include an ice slurry within the first chamber and the second chamber, the ice slurry being at a phase change in a fluid state suitable for delivery to the first chamber and the second chamber through the fluid port.

The ice slurry may include a liquid such as water with one or more additives selected to control a transition temperature of the ice slurry. The ice slurry may include a liquid with one or more additives selected to control a viscosity of the ice slurry. At least one of the first outer surface and the second outer surface may include an insulation chamber to thermally insulate with a layer of air. A second fluid port may provide air to the insulation chamber.

The device may also include at least one tab extending from one of the first chamber and the second chamber for gripping the device with a surgical instrument. The at least one tab may include a non-slip surface texture.

The device may also include a second fluid port for evacuating air from the device during a fill with the ice slurry, where the second fluid port is coupled in fluid communication with the first chamber and the second chamber.

At least one string may be attached to and extending from the device. The device may also include a pocket on the exterior of the device, the pocket shaped and sized to receive a rod for propelling the device into a body cavity through a minimally invasive surgical tool.

Also, the device may comprise at least one visible marking to indicate where to grip the device during a surgical procedure, an orientation of the device, and/or where to cut the device to vent an interior chamber.

The device may further include a second fluid port coupled to one or more air veins within the device, wherein the air veins can be pressurized to control a shape of the device. The device may include a third fluid port coupled in fluid communication with the first chamber and the second chamber, the third fluid port for evacuating air from the device during a fill with the ice slurry.

In another embodiment, the device may include a first fluid port; a first chamber coupled in fluid communication with the fluid port, the first chamber having a first inner surface and a first outer surface, the first outer surface thermally insulated to deter heat flow through the first outer surface; and a second chamber coupled in fluid communication with the fluid port, the second chamber having a second inner surface facing the first inner surface and a second outer surface thermally insulated to deter heat flow through the second outer surface. The second chamber may further comprise one or more baffles mechanically coupling the second inner surface to the second outer surface in order to restrict expansion of the second chamber beyond a predetermined volume, at which predetermined volume additional fluid received through the fluid port will be forcibly directed into the first chamber.

In this embodiment, the first chamber and the second chamber may be shaped and sized to envelop a human organ such as a kidney between the first inner surface and the second inner surface thereof during a surgical procedure. At least one of the first outer surface and the second outer surface may include an insulation chamber to thermally insulate with a layer of air. The fluid port may be suitable for delivering an ice slurry at a phase change in a fluid state to the first chamber and the second chamber.

### DRAWINGS

The invention may be more fully understood with reference to the accompanying drawings wherein:

FIG. 1 is a perspective view of a cooling device.

FIG. 2 is an exploded perspective view of a cooling device.

FIG. 3 is a cross-sectional view of a cooling device.

FIG. 4 is a cross-sectional side view of a cooling device.

FIG. 5 is a cross-sectional side view of a cooling device containing ice slurry and enveloping a kidney.

FIG. 6 is a plan view of a cooling device in an unfolded position.

FIG. 7 is a plan view of a cooling device.

FIG. 8 is a schematic drawing of a system for using the cooling device.

### DETAILED DESCRIPTION

Disclosed herein are devices for cooling an organ such as a kidney during minimally-invasive surgery. It will be understood that the principles disclosed herein may also be usefully employed to control the temperature of an organ in open nephrectomies and transplant procedures while the organ is being inserted into a recipient site. The device and the principles of the invention may more generally be applied in other surgical and non-medical contexts, particularly where thermal control is required over an object that is only accessible through narrow passages. All such variations that would be apparent to one of ordinary skill in the art are intended to fall within the scope of this disclosure.

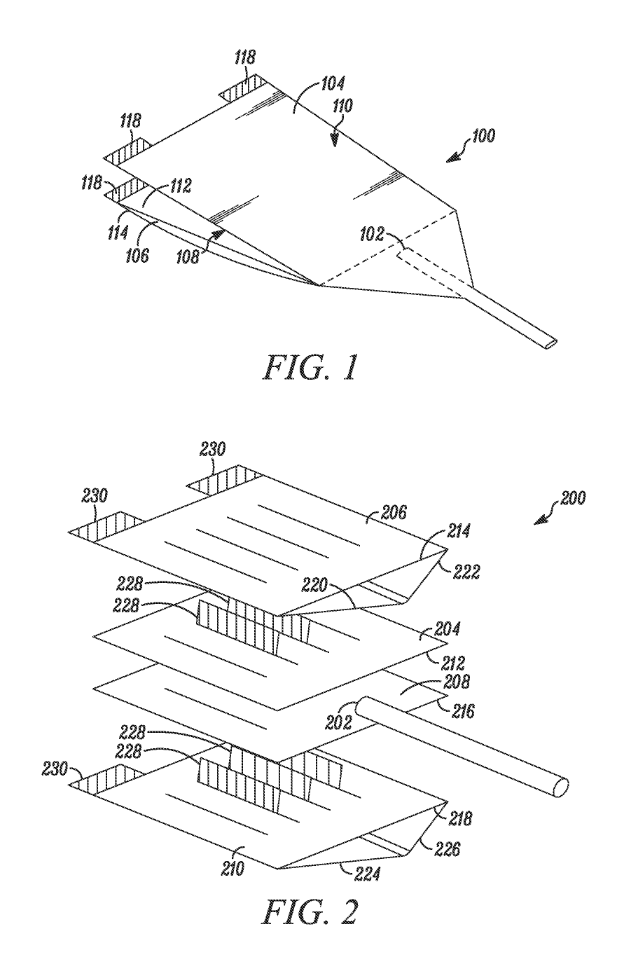

FIG. 1 is a perspective view of a renal cooling device 100. Generally, the device 100 may be a bag or the like having a number of chambers capable of enveloping a human organ such as a kidney between two opposing surfaces. The device 100 may include a fluid port 102, a first chamber 104 (the top chamber), and a second chamber 106 (the bottom chamber). It will be understood that the reference here to two chambers does not imply fluid isolation between the chambers. As will be apparent from the following description, the two chambers may be in direct fluid communication with one another, effectively forming a single, large chamber with two expandable portions that can be filled with a cooling fluid. It should also be appreciated that while two chambers are described, a number of smaller sub-chambers may also or instead be used without departing from the scope of this disclosure.

The first chamber 104 may be coupled in fluid communication with the fluid port 102. The first chamber 104 may have a first inner surface 108 and a first outer surface 110. The second chamber 106 may be coupled in fluid communication with the fluid port 102. The second chamber 106 may have a second inner surface 112 and a second outer surface 114. The second inner surface 112 may face the first inner surface 108 when the device 100 is folded in half, so that the device 100 can envelope an organ or other object to be cooled.

The device 100 may also include one or more tabs 118, which may extend from the first chamber 104 and/or the second chamber 106 in any suitable location(s) to accommodate gripping and handling of the device 100 with surgical tools before, during, and/or after a surgical procedure. At least one tab 118 may include a non-slip surface texture. The tabs 118 may be of sufficient length and/or roughened for easier gripping. The tabs 118 may be the same or different lengths. For example, the tabs 118 extending from the second chamber may be longer than the tabs 118 extending from the first chamber.

FIG. 2 is an exploded perspective view of a cooling device 200 showing the fluid port 202 and layers of sheet material that form the first chamber and second chamber. These layers may includes layers that form a first inner surface 204, a first outer surface 206, a second inner surface 208, and a second outer surface 210. The first inner surface edges may be attached to the corresponding first outer surface edges except for first inner surface edge 212 and first outer surface edge 214. Similarly, the second inner surface edges may be attached to the corresponding second outer surface edges except for second inner surface edge 216 and second outer surface edge 218. The extension areas of the first outer surface 206 and the second outer surface 208, which hold the fluid port 202, may be attached together. In particular, the first extension edges 220, 222 may be attached to the corresponding second extension edges 224, 226, respectively in a manner that surrounds and secures the fluid port 202 in a fluid-tight manner. The first inner surface edge 212 and the second inner surface edge 216 may be attached to each other to form a plenum (along with the extension edges 220-226) that couples the fluid port 202 to the chambers in fluid communication so that, e.g., fluid can be supplied to and removed from the interior chambers.

One or more of baffles 228 may be positioned between the enclosing surfaces, for example between the first inner surface 204 and the first outer surface 206. The baffles 228 may be formed of a pliable material that folds or collapses when the device 200 is uninflated, but that extends perpendicular to the enclosing surfaces when the device 200 is inflated. The baffles 228 may be affixed to the other surfaces using, e.g., ultrasonic welds, heat welds, adhesives, or the like. Any suitable attachment technique may be used provided that it is sufficiently secure to prevent over-inflation of the corresponding chamber under expected use conditions. It will be understood that while three baffles 228 are shown in a top and bottom chamber, the baffles 228 may be used only in a bottom chamber (when deployed for use), and that more or less than three baffles 228 may suitably be employed to similar effect. Additionally, while the baffles 228 are depicted as generally parallel to a path of fluid from the fluid port, and while this orientation reduces resistance to fluid flow into and out of the chambers, the baffles 228 may also or instead be positioned in any orientation, or combination of orientations consistent with delivery of fluid into the chambers.

One or more tabs 230 may extend from device 200, such as from the first outer surface 206 and/or the second outer surface 210. It will be appreciated that more or less tabs 230 may be used, and that the tabs 230 may also or instead extend from other exterior surfaces of the device 200.

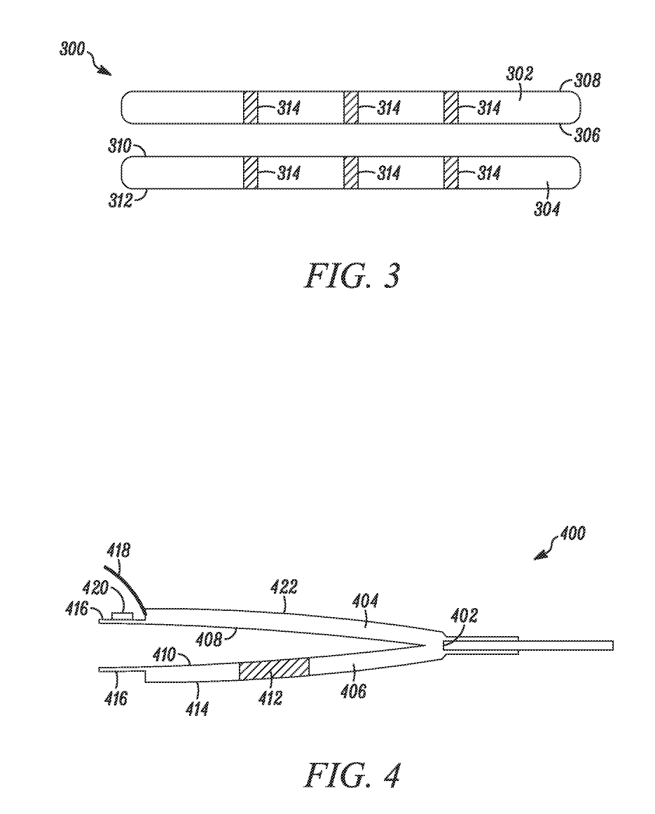

FIG. 3 is a cross-sectional view of a cooling device 300 similar to that of FIG. 2. In this view, the first chamber 302 and the second chamber 304 are separated from one another, such as to accommodate an object for cooling therebetween. In general, the cooling device 300 may include a first inner surface 306 and a first outer surface 308 of the first chamber 302 and a second inner surface 310 and a second outer surface 312 of the second chamber 304. The second chamber 304 may include one or more baffles 314 to control the filling of the second chamber 304 with a fluid. In general, the baffles 314 mechanically couple the second inner surface 310 to the second outer surface 312. The first chamber 302 may also include one or more baffles 314 that mechanically couple the first inner surface 306 to the first outer surface 308.

The one or more baffles 314 may restrict expansion of the second chamber 304 beyond a predetermined volume, at which predetermined volume additional fluid received through the fluid port (not shown) will be forcibly directed into the first chamber 302. Thus, the baffles 314 may prevent the second chamber 304 from overfilling with a fluid by selectively directing addition fluid toward the first chamber 302 once the baffles 314 are extended to their full length (across an interior of the chamber). In this manner, fluid can fill first a bottom chamber (for a fluid having greater density than an organ) and then, when the baffle limit is reached, the top chamber, thus deploying fluid between both chambers without excessive inflation of the lower chamber.

The one or more baffles 314 may be sized and positioned to augment operation of the cooling device 300 in a number of additional ways. In one aspect, the baffles 314 may, when the chambers are inflated, enforce a recessed shape in the surfaces of the cooling device 300 that surround a kidney or other organ in order to retain the organ seated within (e.g., between) the chambers of the cooling device 300. The one or more baffles 314 may similarly prevent the cooling device 300 from inflating from the center toward the outside edges, which may urge a cooled object out of the contained cooling area. The one or more baffles 314 may be specifically arranged to permit greater inflation along a retaining ridge or barrier near a perimeter of the enclosed space. The baffles 314 also advantageously provide a greater thermal surface for cooling than alternatives that employ narrow, circuitous fluid paths. At the same time, the substantially open interior of each chamber, notwithstanding the one or more baffles 314, permits greater fluid mixing and improved heat transfer capabilities (e.g., cooling capabilities) of an enclosed slurry or similar mixture. In addition, the substantially open internal structure mitigates kinks or other blockages that might occur when the cooling device 300 is folded or otherwise compressed during deployment to the target organ.

The one or more baffles 314 may be arranged in various configurations. The baffles 314 may be attached to other materials of the device using, e.g., ultrasonic welds, heat sealing, biocompatible adhesives, and/or any adhesives consistent with maintaining a bond in the presence of the various cooling mixtures contemplated herein. Any biocompatible plastic, such as polyurethane film, or similar sheet material may be used. In one aspect, constructing the baffles 314 from the same, or a similar, material as the chamber walls permits the cooling device 300, when uninflated with a cooling medium, to be folded, rolled, or otherwise collapsed into a compact form for deployment through minimally invasive surgical tools.

FIG. 4 is a cross-sectional side view of another embodiment of a cooling device 400. The fluid port 402 may be coupled to the device in the vicinity where the first chamber 404 and the second chamber 406 are adjacent such that the fluid port 402 is in fluid communication with the first chamber 404 and the second chamber 406. FIG. 4 shows the attachment of the first inner surface 408 and the second inner surface 410 at the edge of these respective surfaces that is closest to the fluid port 402. A baffle 412 as shown from the side in FIG. 4 may mechanically couple the second inner surface 410 to the second outer surface 414 of the second chamber 406.

The device 400 may also include at least one string 418 attached to and extending from the device 400 for manipulating the device 400. A string 418 may extend from the first outer surface 422 of the first chamber 404 or any other portion of the device 400. The string 418 may be used to hold the device 400 together and for control and retraction. In particular, the string 418 may be used to hold back the first chamber 404 so that second chamber 406 can be manipulated.

The device 400 may further include a pocket 420 on the exterior of the device 400, where the pocket 420 is shaped and sized to receive a rod for propelling the device 400 into a body cavity through a minimally invasive surgical tool. For example, the device may have a pocket 420 on a tab 416 to receive a stiff rod that can push the device 400 through a trocar. The pocket 420 may be positioned on a tab 416 extending from the first outer surface 422 of the first chamber 406, or the pocket 420 may be positioned on any other portion of the device 400.

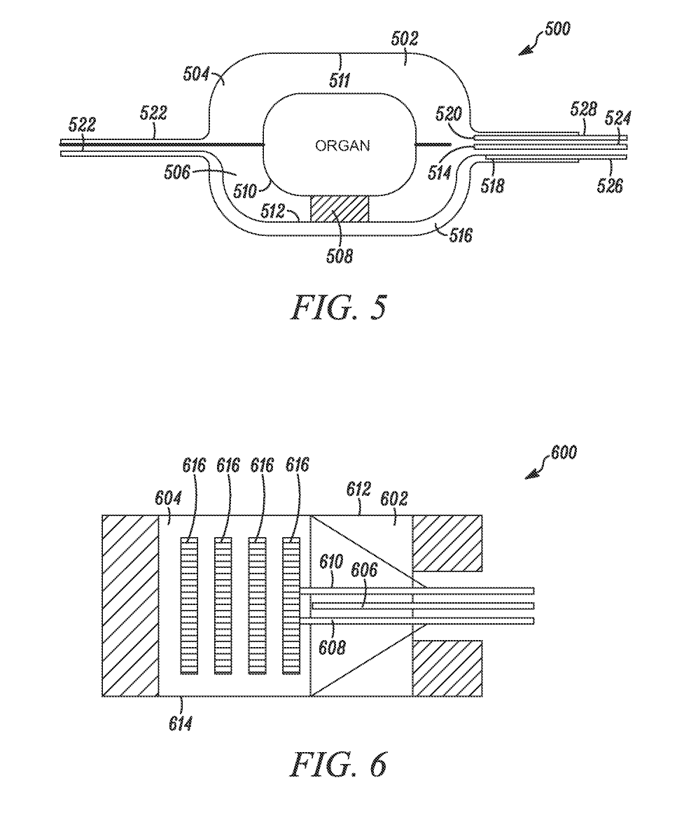

FIG. 5 is a cross-sectional side view of a cooling device 500 enveloping a kidney. At least one of a first chamber 504 and a second chamber 506 may contain an ice slurry 502 or other cooling fluid that cools the kidney. Suitable ice slurries for organ cooling as contemplated herein are discussed in greater detail below. The first chamber 504 may be positioned on the top portion of the kidney while the second chamber 506 may be positioned underneath the kidney. Having one chamber on each side of the organ ensures substantial surface area contact with the organ. One or more baffles 508 in the second chamber 506 may mechanically couple the second inner surface 510 to the second outer surface 512 as described above.

The device 500 may also include a first fluid port 514 coupled in fluid communication with the first chamber 504 and the second chamber 506, a second fluid port 518 coupled in fluid communication with an insulation layer 516, and a third fluid port 520 coupled in fluid communication with the first chamber 504.

A substance may be delivered through the first fluid port 514 to the first chamber 504 and/or the second chamber 506. The first fluid port 514 may be attached to a tube 524 for delivering the substance though the first fluid port 514. The first tube 524 may be coupled in fluid communication with the first fluid port 514.

The device 500 may also include a second fluid port 518 coupled in fluid communication with the insulating layer 516 formed in a space between a first outer surface 511 (which is exposed to an environment) and the second outer surface 512 of the cooling chamber. The second fluid port 518 may be attached to a second tube 526 through which air or other insulating gas, fluid, or the like can be delivered. A second tube 526 may be coupled in fluid communication with the second fluid port 518. The second tube 526 may be smaller than the first tube 524.

It may be desirable to remove excess air trapped in the first chamber 504 so that the first chamber 504 may be completely filled with ice slurry 502. The second tube 526 may also be used for removal of air that is trapped in the device 500 during the slurry filling process or a third fluid port 520 may be used. The first chamber 504 of the device 500 may include a third fluid port 520 for evacuating air from the device 500, which may include a one-way valve, check valve, or the like to prevent re-entry of air through the third fluid port 520. The third fluid port 520 may be coupled in fluid communication with the first chamber 504 and/or the second chamber 506. The third fluid port 520 may also be coupled to a third tube 528. Alternatively, an additional tube may be added to the inside of the first tube 524 to act as a relief for air that becomes trapped in the device 500 during slurry delivery.

The tubes 524, 526, 528 may have any interior and exterior diameter, and may be fabricated from any suitable material, for use in minimally-invasive surgical procedures as contemplated herein. By way of example, the tubes 524, 526, 528 may be formed from polyurethane or any other biocompatible polymer or other material. One or more of the tubes 524, 526, 528 may be clear in order to permit visual inspection of fluids passing therethrough.

The substance delivered to the device may be an ice slurry at a phase change in a fluid state suitable for delivery to the first chamber and the second chamber through the fluid port. In this state, the ice slurry is flowable enough to pass through the fluid port, while providing favorable heat transfer characteristics for cooling an organ or the like. Advantageously, the ice slurry, when in this transitional phase, maintains a freezing temperature (around zero degrees Celsius for water-based slurries, with variations according to additives) until all of the ice in the slurry melts. Phase change material takes advantage of the large latent heat of fusion of water (334 kJ/kg) which can reduce the amount of fluid required for organ cooling as contemplated herein, as well as reducing or eliminating the need for active circulation of the fluid.

The amount of ice slurry used may depend upon the nature of the environment and properties of the object to be cooled. For example, the amount of ice slurry may account for heat transfer to the target organ, temperature and heat transfer properties of the body cavity containing the organ, and the adjacent tissue. In one aspect, the cooling device 500 may provide an inflated interior volume of about 800 ml, thus accommodating about 800 ml of ice slurry.

The ice slurry may include a liquid such as water with one or more additives. The additives may be selected to control a transition temperature of the ice slurry or to control a viscosity of the ice slurry. The additive may increase the viscosity of the water or a resulting ice slurry, causing the ice particles to remain in suspension and yielding a continuous phase composition. For example, the ice slurry may be a mixture of ice, water, and sugar (all biocompatible materials). A suitable mixture may comprise a solution of 54.9% ice and 45.1% water and 17.3% sugar by weight. More generally, the composition of ice slurries suitable for organ cooling is well known. Numerous example are provided, by way of illustration and not limitation, in U.S. Pat. No. 6,413,444 to Kasza, incorporated by reference herein in its entirety.

The first chamber and the second chamber may be shaped and sized to envelop a human organ between the first inner surface and the second inner surface thereof during a surgical procedure. The human organ may be a kidney. The device may be long enough to cover the entire organ. In addition, the device may be shaped so that it can be folded and inserted into a deployment device such as a trocar and then guided back into the deployment device and/or pulled through an incision upon removal from the body. In particular, the device may be designed to be rolled for its initial deployment, and pushed into the body cavity through a tube. For example, it may be sized and shaped to be folded for fit into a 15 mm trocar or smaller for delivery to a body cavity through an incision. The device may be fabricated from durable, tear-resistant materials in order to facilitate deployment through the trocar. The device may be advantageously constructed substantially completely from thin sheets of pliable plastic or similar material amenable to folding, rolling, or the like for such deployment.

The device may have any suitable thickness that would allow the device to be folded, rolled, and/or otherwise compressed for insertion into a delivery device such as an endoscopic trocar. In one embodiment, the sheets of material may have a thickness of any readily available and suitable plastic sheet material, such as 0.0254 mm, 0.0508 mm, 0.0765 mm, 0.1016 mm, 0.127 mm, 0.1778 mm, and 0.254 mm (corresponding to 1, 2, 3, 4, 5, 7, and 10 thousandths of an inch). More generally, any standard or non-standard thickness of material suitable for construction into a layered device as described herein and collapsible for deployment through a minimally invasive surgical tool may be used for fabrication of the cooling device and/or baffles and other internal and external structures.

A combination of thicknesses may be used. The outer surfaces and inner surfaces of the chambers may have the same or different thickness. For example, a thinner film of material may be used on the inner surfaces (for better heat transfer) and thicker film of material may be used on the outer surfaces of the chambers (for better insulation) and on the tabs where the surgeon might grasp the device. In one embodiment, the outer surfaces may have a thickness of 0.127 mm, while the inner surfaces may have a thickness of 0.051 mm.

In general, the chambers may be fabricated from transparent or semi-transparent materials that permit viewing of cooling mixtures within the device. For example, the chambers may be made from a plastic material such as polyurethane films or low density polyethylene. One or more materials may be used to make the chambers. The first chamber and second chamber may be made from separate layers of material that may be attached or sealed together.

The first outer surface may be thermally insulated to deter heat flow through the first outer surface. Also, the second outer surface may be thermally insulated to deter heat flow through the second outer surface. The device may be thermally insulated using any suitable insulating material, and/or with a layer of air as discussed above. This approach advantageously permits use of a relatively thick insulating layer of air, while permitting evacuation of the insulating air layer for insertion and removal of the cooling device 500.

The insulation layer may provide insulation from heat generated from other body parts such as the abdomen, reduce cooling of surrounding tissue, and slow melting of the ice slurry or other substance in the device. The insulation layer, and in particular, an inflatable air insulation layer, which can be highly pressurized to impose shape, may also provide structural rigidity to the device to aid in manipulation of the device in the body and around an organ. An air-inflatable insulation layer may aid in unfurling the device upon deployment in the body.

The insulation layer may be of any suitable thickness to reduce the melting of the ice slurry or other substance. In one embodiment, an insulation layer on the second outer surface has a thickness of 1 cm.

FIG. 6 is a plan view of a device 600 in an unfolded position showing a first outer surface 602 and a second outer surface 604 of the device 600. An extended portion including a first fluid port 606, a second fluid port 608, and a third fluid port 610 may extend over the first outer surface 602 of a first chamber 612. A second chamber 614 may include a plurality of air veins 616 on a second outer surface 604.

The device 600 may include air cavities that, when pressurized, impose a desired shape on the device 600. This may for example include causing the device 600 to unroll from a compressed state, or imposing a desired shape for use in organ cooling. For example, the device 600 may include one or more air veins 616 or ribs that can be pressurized to control a shape of the device 600. The air veins 616 may be formed as part of an insulation layer surrounding the first chamber or the second chamber as described above, or as a different and/or additional layer. Each one of the air veins 616, when pressurized, may provide lateral stiffness along its length. The second fluid port 608 may be coupled in fluid communication to the one or more air veins 616 within the device 600 in order to provide air or other pressurizing gas/fluid to inflate the air veins 616. The second fluid port 608 may also be used for air removal, or the device 600 may include a third fluid port 610 for air removal. The third fluid port 610 (or the second fluid port 608 may also be coupled to an air vein 614 about a perimeter of the device 600. In one aspect, a one way valve may be used to hold pressure within the air veins 616 independent of forward pressurization through the fluid port(s). In another aspect, the device 600 may be cut, e.g., with a surgical tool, in order to deflate the air veins 616 for withdrawal of the device. An area of the device 600 separate from the chambers of cooling fluid may be visually marked and provided for this purpose, so that the air can be removed in this manner without risk of cutting the chambers of cooling fluid and spilling cooling fluid into a body cavity.

In one aspect, the air veins 616 may be fabricated from tubes or the like adhered to other layers of the device 600. In another aspect, an additional layer of pliable sheet material may be adhered (or otherwise attached) to the device 600 in a pattern that defines a desired network of air veins 616 for use as described above.

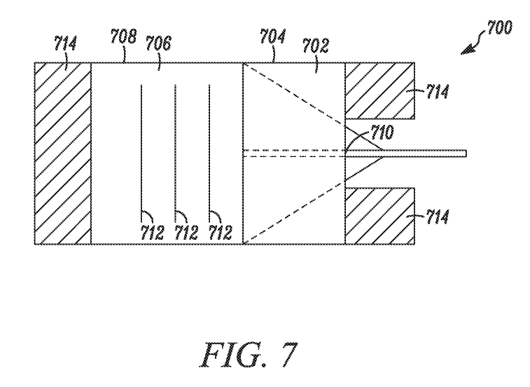

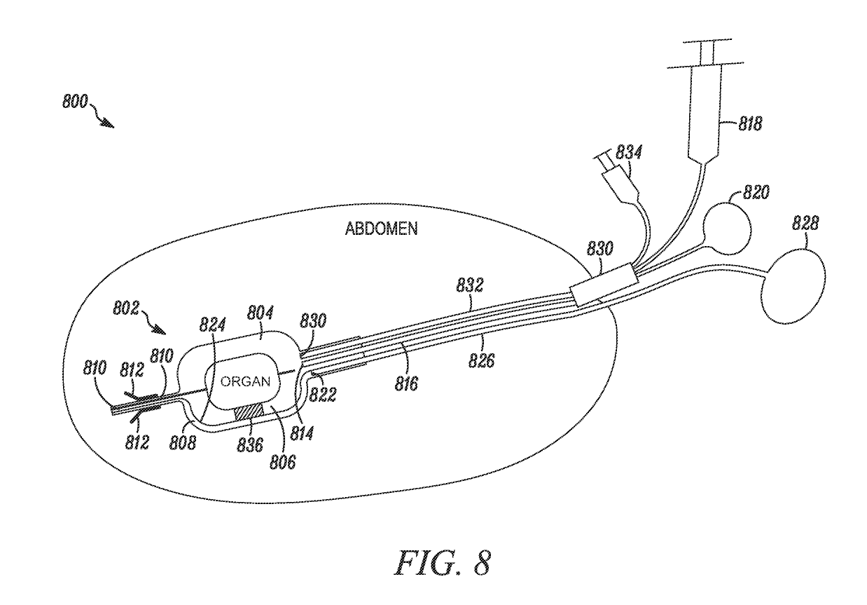

FIG. 7 is a plan view of a device 700 in an unfolded position showing a first inner surface 702 of a first chamber 704 and a second inner surface 706 of a second chamber 708. An extended portion including a fluid port 710 lies under the first chamber 704. The device 700 may include at least one visible marking 712 to aid with handling and use of the device 700. For example, the visible marking 712 may indicate where to grip the device 700 during a surgical procedure, or may indicate an orientation of the device 700 relative to the organ for proper placement and use. The visible marking 712 may also or instead show where to cut the device 700 to vent an interior chamber such as an air insulation chamber or the reinforcing veins described above. As another example, a pattern of lines on the tabs 714 may be used as visible markings to indicate where to grip the device 700 during a surgical procedure.

The visible markings 712 may include words to indicate a use such as “cut here” or “grip here”, and/or the visible markings 712 may indicate orientation such as “top” or “bottom”. The visible markings 712 may also or instead include color coding such as different colors or patterns to distinguish the first chamber from the second chamber, the outer surfaces from the inner surfaces, or the tabs from the rest of the device.

FIG. 8 is a schematic drawing of an example of a system 800 for using the device 802 during minimally invasive surgery. In use, the device 800 may be deployed to a target organ, positioned around the organ, filled with a substance and left in place to cool the organ, drained of the substance, and then removed from the organ.

The system 800 may include a cooling device 802 having a first chamber 804, a second chamber 806, an insulation layer 808, one or more tabs 810 that may be secured by a clamp 812, and three fluid ports 814, 822, 830. The first fluid port 814 may be coupled in fluid communication with the first chamber 804 and the second chamber 806 of the device 802 and further attached to a first tube 816, which may be attached to a fluid delivery device 818 and a deployment device 820. The second fluid port 822 may be coupled in fluid communication with the insulation layer 808 on the second outer surface 824 of the second chamber 806 and with a second tube 826 which may be attached to an air pump 828 for inflating the insulation layer 808. The third fluid port 830 may be coupled in fluid communication with the first chamber 804 and the second chamber 806 and with a third tube 832 that may be connected to an air removal device 834. The system 800 and use of the cooling device 802 are now described in greater detail.

The deployment device 820 may including any suitable device(s) for delivering the cooling device 802 during a minimally invasive surgery, including without limitation a trocar for endoscopic or laparoscopic surgery. Thus for example the deployment device 820 may include a trocar 830 to deliver the device 802 through an incision to an insufflated abdomen, along with the first tube 816 and the first fluid port 814 that couple the cooling device 802 the fluid delivery device 818 for supply of a cooling mixture such as an ice slurry. The fluid delivery device 818 may include a large volume syringe, or an automatic or manually controlled pump, along with any equipment required to maintain the cooling mixture in a suitable state for use in organ cooling (e.g., refrigeration, mixing, etc.). The deployment device 820 may deliver the cooling device 802 to a body cavity through an internal channel of the trocar 830, along with the first tube 816 and the first fluid port 814. The trocar may be a 12 mm or 15 mm trocar.

The cooling device 802 may include an extra layer for protection during deployment, such as a deployment sleeve or a sheath before, which may include a receptacle to secure a driving rod. The cooling device 802 may be rolled into a cylinder or other compact configuration along its long axis, placed within a steel sheath with a 12 mm outer diameter and then delivered through a 12 mm trocar 830. Once inserted into the body cavity, the cooling device 802 can be pushed through the sleeve or sheath and the sleeve or sheath may be removed from the trocar 830, or the sheath may include a mechanical stop to prevent forward motion of the sheath beyond an end of the trocar.

Alternatively, the cooling device 802 may be rolled around a thin metal rod and then deployed through the trocar 830. The cooling device 802 may have a pocket or similar receptacle on the tab or at any other convenient location to receive a rod that can apply mechanical force to drive the cooling device 802 through the trocar 830 to the surgical site. The rod may also provide a structure to support rolling or other configuration of the cooling device 802 for deployment, and the rod may add stiffness to the cooling device 802 itself so that it can be delivered through the trocar 830.

In another aspect, the first tube 816 that delivers the ice slurry can also be constructed with sufficient rigidity to guide the cooling device 802 through the trocar 830, or a rod may be inserted into the first tube 816 for deployment. Any other method suitable for delivering medical devices may be used to deploy the device 802.

After the cooling device 802 is deployed, the cooling device 802 may be positioned and wrapped around a target organ such as a kidney using standard lapaoscopic or endoscopic instruments. For example, the cooling device 802 may be positioned in an insufflated abdomen and around a kidney. The tabs 810 may provide a convenient gripping surface for handling and positioning the cooling device 802, such as for drawing the cooling device 802 (in its uninflated form) underneath and around an organ. In another aspect, surface reinforcement may be provided in various areas, such as along edges or on corners of the cooling device 802 (along with corresponding visual indicators as described above) to facilitate gripping with endoscopic surgical tools.

An insulation layer 808 may aid in unfurling and positioning the device 802 by pressurization with air or other suitable gas, or additional air channels may be provided that can be inflated to impose a desired shape to the cooling device 802. The second tube 826 may be attached to an air pump 828 that delivers air to the insulation layer 808. This may be a manual air pump, an automated electromechanical air pump, or any other suitable hardware for delivering a gas at a controlled pressure or volume to the insulation layer 808 and/or additional air channels. The air pump 828 may deliver the air (or other gas) using a one-way valve. For example, the insulation layer 808 may be inflated and deflated with a squeeze bulb pump and relief valve such as found on a blood-pressure cuff, or any other similar manual or machine assisted pressure control mechanism(s). Once the cooling device 802 is unrolled and in a flat position (as shown in FIG. 6), the air may be evacuated using an air removal device 834 (which may be the same as the air pump 828), or permitted to passively evacuate during use.

When deployed, the second chamber 806 may be positioned underneath a posterior surface of a kidney. A retraction string may be affixed to the cooling device 802 at any suitable location and used to pull the first chamber back while the kidney is being positioned on the inner surface of the second chamber 806. The tabs 810 can also or instead be used to position the cooling device 802.

Once the second chamber 806 is placed under the kidney, the first chamber 804 may be folded over the kidney and the tabs 810 extending from the first chamber 804 and the second chamber 806 may be clamped together using surgical clamps 812 or the like to envelop and provide substantial surface area contact with the kidney. One clamp 812 may be used for each side of the device 802. In a renal surgery, the clamps 812 may be advantageously positioned on an outside of the cooling device 802 to avoid interference with the renal artery.

After the cooling device 802 is positioned around the organ, the cooling device 802 may be filled with a suitable amount of a fluid such as an ice slurry using the fluid delivery device 818. It will be noted that for a typical organ, the organ will be less dense than the cooling ice slurry used. Thus the cooling device 802 will fill around the organ until there is sufficient mass to displace (and thus float) the organ, at which time fluid will flow steadily to a chamber beneath the organ. The baffles 836 prevent this bottom chamber from filling beyond a predetermined volume, at which time additional fluid is forced (by the volume-limited lower chamber) into a top chamber which covers an upper surface of the organ. Thus as discussed above, the one or more baffles 836 in the second chamber 806 may limit fluid flow into the second chamber 806 forcing the remaining fluid to the first chamber 804 to completely surround the kidney.

The third tube 832 may be attached to an air removal device 834 that may be used to remove excess air from the cooling device 802 before or during delivery of the ice slurry. The air removal device 834 may be a syringe. As noted above, air removal may instead be accomplished through the air supply feed. An additional tube (which may be the third tube 832) may be provided to vent excess air from an interior of the cooling device 802 during filling with a cooling mixture.

Once the cooling device 802 is filled, the cooling device 802 may remain in place for a desired amount of time to cool the organ. For example, the device 802 containing the ice slurry therein may remain on the kidney for up to 20 minutes to cool the kidney to below 20° C. The insulation layer 808 may be filled with air after the cooling device 802 is positioned around an organ to slow melting of the ice slurry by insulating the ice slurry from warmer surrounding tissue.

After cooling the organ and/or completion of a surgical procedure, the ice slurry or other cooling fluid may be removed from the cooling device 802 using any suitable method. For example, the cooling device 802 may be unclamped and a standard hospital suction system may be attached to the first tube 816 to remove most of the ice slurry from the device 802. Any remaining fluid may be drained by cutting a slit near the tabs 810 or other portion of the cooling device 802, with an understanding that such fluid should be in a form safe for disposition within the body.

After the cooling device 802 is drained, the cooling device 802 may be retracted from the body through the incision using any suitable method for removing a medical device during minimally invasive surgery. The cooling device 802 may be grasped and pulled through the trocar 830, or the trocar 830 may be removed first and then the cooling device 802 may be extracted through the trocar incision. A string or other protrusion may be provided to facilitate grasping for withdrawal. In one aspect, the string may be deployed with and attached to the cooling device 802 on one end, with the other end remaining threaded through the trocar 830 and freely accessible outside the body for convenient extraction of the cooling device 802. The cooling device 802 may be removed from the body before or after surgery. For example, if a tumor on an organ is on the top of the organ, the first chamber 804 may be pulled back during surgery and the cooling device 802 left in place with the top of the organ exposed during the surgical procedure.

It will be appreciated that the devices, methods and systems described above are set forth by way of example and not of limitation. Numerous variations, additions, omissions, and other modifications will be apparent to one of ordinary skill in the art. While particular embodiments of the present invention have been shown and described, it will be apparent to those skilled in the art that various changes and modifications in form and details may be made without departing from the spirit and scope of the invention as defined by the following claims. The claims that follow are intended to include all such variations and modifications that might fall within their scope, and should be interpreted in the broadest sense allowable by law.

What is claimed is:

1. A device comprising:
a fluid port;
a first chamber coupled in fluid communication with the fluid port, the first chamber having a first inner surface and a first outer surface, the first outer surface thermally insulated to deter heat flow through the first outer surface; and
a second chamber coupled in fluid communication with the fluid port, the second chamber having a second inner surface facing the first inner surface and a second outer surface thermally insulated to deter heat flow through the second outer surface, the second chamber further comprising one or more baffles mechanically coupling the second inner surface to the second outer surface in order to restrict expansion of the second chamber beyond a predetermined volume, at which predetermined volume additional fluid received through the fluid port will be forcibly directed into the first chamber.

2. The device of claim 1, wherein the first chamber and the second chamber are shaped and sized to envelop a human organ between the first inner surface and the second inner surface thereof during a surgical procedure.

3. The device of claim 2, wherein the human organ is a kidney.

4. The device of claim 1, further comprising an ice slurry within the first chamber and the second chamber, the ice slurry at a phase change in a fluid state suitable for delivery to the first chamber and the second chamber through the fluid port.

5. The device of claim 4, wherein the ice slurry includes a liquid with one or more additives selected to control a transition temperature of the ice slurry.

6. The device of claim 5, wherein the liquid is water.

7. The device of claim 4, wherein the ice slurry includes a liquid with one or more additives selected to control a viscosity of the ice slurry.

8. The device of claim 1, wherein at least one of the first outer surface and the second outer surface includes an insulation chamber to thermally insulate with a layer of air.

9. The device of claim 8, further comprising a second fluid port to provide air to the insulation chamber.

10. The device of claim 1, further comprising at least one tab extending from one of the first chamber and the second chamber for gripping the device with a surgical instrument.

11. The device of claim 10, wherein the at least one tab includes a non-slip surface texture.

12. The device of claim 1, further comprising a second fluid port coupled in fluid communication with the first chamber and the second chamber, the second fluid port for evacuating air from the device during a fill with an ice slurry.

13. The device of claim 1, further comprising at least one string attached to and extending from the device.

14. The device of claim 1, further comprising a pocket on the exterior of the device, the pocket shaped and sized to receive a rod for propelling the device into a body cavity through a minimally invasive surgical tool.

15. The device of claim 1, further comprising at least one visible marking to indicate where to grip the device during a surgical procedure.

16. The device of claim 1, further comprising at least one visible marking to an orientation of the device.

17. The device of claim 1, further comprising at least one visible marking to indicate where to cut the device to vent an interior chamber.

18. The device of claim 1, further comprising a second fluid port coupled to one or more air veins within the device, wherein the air veins can be pressurized to control a shape of the device.

19. The device of claim 1, further comprising a second fluid port coupled to one or more air veins within the device, wherein the air veins can be pressurized to control a shape of the device, and a third fluid port coupled in fluid communication with the first chamber and the second chamber, the third fluid port for evacuating air from the device during a fill with an ice slurry.

20. A device comprising:
a first fluid port;
a first chamber coupled in fluid communication with the fluid port, the first chamber having a first inner surface and a first outer surface, the first outer surface thermally insulated to deter heat flow through the first outer surface; and
a second chamber coupled in fluid communication with the fluid port, the second chamber having a second inner surface facing the first inner surface and a second outer surface thermally insulated to deter heat flow through the second outer surface, the second chamber further comprising one or more baffles mechanically coupling the second inner surface to the second outer surface in order to restrict expansion of the second chamber beyond a predetermined volume, at which predetermined volume additional fluid received through the fluid port will be forcibly directed into the first chamber,
the first chamber and the second chamber shaped and sized to envelop a human organ between the first inner surface and the second inner surface thereof during a surgical procedure;
at least one of the first outer surface and the second outer surface including an insulation chamber to thermally insulate with a layer of air; and
the fluid port being suitable for delivering an ice slurry at a phase change in a fluid state to the first chamber and the second chamber.

21. The device of claim 20, wherein the organ is a kidney.
